(19) United States
(12) Patent Application Publication
(10) Pub. No.: US 2010/0159474 A1
(43) Pub. Date: Jun. 24, 2010

(54) PROGNOSIS AND RISK ASSESSMENT IN PATIENTS SUFFERING FROM HEART FAILURE BY DETERMINING THE LEVEL OF ADM AND BNP

(75) Inventors: Andreas Bergmann, Berlin (DE); Oliver Hartmann, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: B.R.A.H.M.S. AKTIENGESELLSCHAFT, HENNIGSDORF (DE)

(21) Appl. No.: 12/613,891

(22) Filed: Nov. 6, 2009

Related U.S. Application Data
(60) Provisional application No. 61/113,649, filed on Nov. 12, 2008.

(30) Foreign Application Priority Data
Nov. 11, 2008 (EP) 08 16 8816.0

(51) Int. Cl. G01N 33/53

(52) U.S. Cl. 435/7.1; 436/501

(57) ABSTRACT

The present invention relates to a method for prognosis of an outcome or assessing the risk of a patient suffering from heart failure and/or shortness of breath, comprising the determination of the levels of ADM or fragments thereof or its precursor or fragments thereof and BNP or fragments thereof or its precursor or fragments thereof in said sample of said patient.

This application claims the benefit of the filing date of U.S. Provisional Application Ser. No. 61/113,649 filed Nov. 12, 2008.

### FIELD OF THE INVENTION

The present invention is in the field of clinical diagnostics. Particularly the present invention relates to outcome prognosis and risk assessment in patients suffering from heart failure and/or shortness of breath by determination of the level of marker peptides.

### BACKGROUND OF THE INVENTION

Heart failure (HF), also termed congestive heart failure (CHF) is a cardiac condition that occurs when a problem with the structure or function of the heart impairs its ability to supply sufficient blood flow to meet the body's needs. It can cause a large variety of symptoms, particularly shortness of breath (SOB) at rest or during exertion and/or fatigue, signs of fluid retention such as pulmonary congestion or ankle swelling, and objective evidence of an abnormality of the structure or function of the heart at rest. However, some patients can be completely symptom free and asymptomatic structural or functional abnormalities of the heart are considered as precursors of symptomatic heart failure and are associated with high mortality (Wang et al. 2003. Circulation 108: 977-82). Heart failure is a common disease: more than 2% of the U.S. population, or almost 5 million people, are affected and 30 to 40% of patients die from heart failure within 1 year after receiving the diagnosis (McMurray J. J., Pfeffer M. A. 2005. Lancet 365: 1877-89). Heart failure is often undiagnosed due to a lack of a universally agreed definition and challenges in definitive diagnosis, particularly in the early stage. With appropriate therapy, heart failure can be managed in the majority of patients, but it is a potentially life threatening condition, and progressive disease is associated with an overall annual mortality rate of 10%. It is the leading cause of hospitalization in people older than 65 years (Haldemann G. A. et al. 1999. Am Heart J 137: 352-60). As a consequence, the management of heart failure consumes 1-2% of total health-care expenditure in European countries (Berry et al. 2001. Eur J Heart Fail 3: 749-53).

### DESCRIPTION OF THE INVENTION

A subject of the present invention is the provision of a method for the prognosis of an outcome or assessing the risk of a patient suffering from heart failure and/or shortness of breath, comprising the following steps:
a. providing a sample from said patient,
b. determining the level of ADM or fragments thereof or its precursor or fragments thereof in said sample,
c. determining the level of BNP or fragments thereof or its precursor or fragments thereof, in said sample,
d. correlating the level of said ADM or fragments thereof or its precursor or fragments thereof and BNP or fragments thereof or its precursor or fragments thereof with the prognosis of an outcome or assessing the risk for said patient.

Said outcome or said risk regards may for instance regard the survival and/or a functional outcome.

Heart failure herein preferably relates to acute heart failure (AHF).

In a preferred embodiment of the invention the outcome after 90 days, preferably after 60 days, more preferably after 45 days, most preferably after 30 days is predicted.

The invention also provides a method for the stratification of a patient into risk groups said patient suffering from heart failure and/or shortness of breath and said method comprising the steps as described above.

Preferably herein ADM or fragments thereof or its precursor or fragments thereof is mid-regional proAdrenomedullin (MR-proADM).

It is also preferred that BNP or fragments thereof or its precursor or fragments thereof is NT-proBNP.

In a particular embodiment of the inventive methods the level of ADM or fragments thereof or its precursor or fragments thereof and BNP or fragments thereof or its precursor or fragments thereof are differently weighted.

The functional outcome may be determined as ranking or the degree of severity of the disease e.g. the state of health of a patient after a defined time.

In a preferred embodiment, additionally at least one clinical parameter is determined selected from the group comprising: age, gender, systolic blood pressure, diastolic blood pressure, antihypertensive treatment, body mass index, heart rate, temperature, presence of diabetes mellitus and current smoking habits, most preferably selected from the group consisting of heart rate, temperature, body mass index, systolic blood pressure and diastolic blood pressure.

In some particular embodiments other laboratory parameters may additionally be determined, e.g. the level of further prognostic markers, particularly other peptide hormones and fragments thereof or precursors or fragments thereof. Further prognostic markers may be selected from the group comprising troponin, myeloperoxidase, CRP, neopterin, GDF-15, ST2, cystatin-C, as well as the following peptides in form of their mature peptides, precursors, pro-hormones and associated prohormone fragments: atrial natriuretic peptide, endothelins, vasopressin.

The invention relates in a particular embodiment to a method for the prognosis of an outcome or assessing the risk of a patient suffering from heart failure and/or shortness of breath according to the methods described above, wherein the levels of ADM or fragments thereof or its precursor or fragments thereof and BNP or fragments thereof or its precursor or fragments thereof either alone or in conjunction with other prognostically useful laboratory or clinical parameters are used for the prediction of an outcome or assessing the risk of a patient suffering from heart failure and/or shortness of breath by a method which may be selected from the following alternatives:
Comparison with the medians of the levels of ADM or fragments thereof or its precursor or fragments thereof and BNP or fragments thereof or its precursor or fragments thereof in an ensemble of pre-determined samples in a population of patients suffering from heart failure and/or shortness of breath,
Comparison with a quantile of the levels of ADM or fragments thereof or its precursor or fragments thereof and BNP or fragments thereof or its precursor or fragments thereof in an ensemble of pre-determined samples in a population of patients suffering from heart failure and/or shortness of breath,
Calculation based on Cox Proportional Hazards analysis or by using Risk index calculations such as the NRI (Net Reclassification Index) or the IDI (Integrated Discrimination Index).

In a further embodiment the present invention relates to a method for the prognosis of an outcome or assessing the risk of a patient suffering from heart failure and/or shortness of breath, comprising the following steps:
a. providing a sample from said patient,
b. determining the level of ADM or fragments thereof or its precursor or fragments thereof in said sample,
c. correlating the level of said ADM or fragments thereof or its precursor or fragments thereof with the prognosis of an outcome or assessing the risk for said patient.

When using ADM as marker it is a preferred embodiment of the methods according to the invention to determine troponin in addition to ADM or fragments thereof or its precursor of fragments thereof. ADM adds significantly troponin and troponin adds significants to MR-ADM.

It is preferred in this particular embodiment that the short time outcome, i.e. the outcome within or after 45 days, 40 days, 35 days, 30 days, 25 days, 20 days, 15 days, 10 days or 5 days, preferably after 30 days is predicted. In another preferred embodiment of the invention the outcome after 90 days is predicted.

The invention also relates to a kit for the prognosis of an outcome or assessing the risk of a patient suffering from heart failure and/or shortness of breath comprising at least one capture probe directed against ADM or fragments thereof or precursors or fragments thereof and at least one capture probe directed against BNP or fragments thereof or precursors or fragments thereof.

Preferably, the capture probe directed against ADM or fragments thereof or precursors or fragments thereof is directed against MR-proADM.

The capture probe directed against BNP or fragments thereof or precursors or fragments thereof is preferably directed against NT-proBNP.

The invention also relates to the use of the described methods and kits for the prognosis of an outcome or assessing the risk of a patient suffering from heart failure and/or shortness of breath. Furthermore, the invention relates to the use of the methods and kits for the monitoring of therapy in a patient suffering from heart failure and/or shortness of breath.

The levels of the markers as obtained by the methods or the use of the methods according to the present invention may be analyzed in a number of fashions well known to a person skilled in the art. For example, each assay result obtained may be compared to a “normal” value, or a value indicating a particular disease or outcome. A particular diagnosis/prognosis may depend upon the comparison of each assay result to such a value, which may be referred to as a diagnostic or prognostic “threshold”. In certain embodiments, assays for one or more diagnostic or prognostic indicators are correlated to a condition or disease by merely the presence or absence of the indicator(s) in the assay. For example, an assay can be designed so that a positive signal only occurs above a particular threshold concentration of interest, and below which concentration the assay provides no signal above background.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy) and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art. See, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In certain embodiments, particular thresholds for one or more markers in a panel are not relied upon to determine if a profile of marker levels obtained from a subject are indicative of a particular diagnosis/prognosis. Rather, the present invention may utilize an evaluation of a marker panel “profile” as a unitary whole. A particular “fingerprint” pattern of changes in such a panel of markers may, in effect, act as a specific diagnostic or prognostic indicator. As discussed herein, that pattern of changes may be obtained from a single sample, or from temporal changes in one or more members of the panel (or a panel response value). A panel herein refers to a set of markers.

As described herein after, a panel response value is preferably determined by plotting ROC curves for the sensitivity (i.e. true positives) of a particular panel of markers versus 1-(specificity) (i.e. false positives) for the panel at various cut-offs. In these methods, a profile of marker measurements from a subject is considered together to provide a global probability (expressed either as a numeric score or as a percentage risk) of a diagnosis or prognosis. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/5% of a given measurement.

The skilled artisan will understand that associating a diagnostic or prognostic indicator, with a diagnosis or with a prognostic risk of a future clinical outcome is a statistical analysis. For example, a marker level of greater than X may signal that a patient is more likely to suffer from an adverse outcome than patients with a level less than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In yet other embodiments, multiple determinations of diagnostic or prognostic markers can be made, and a temporal change in the marker can be used to determine a diagnosis or prognosis. For example, a marker concentration in a subject sample may be determined at an initial time, and again at a second time from a second subject sample. In such embodiments, an increase in the marker from the initial time to the second time may be indicative of a particular diagnosis, or a particular prognosis. Likewise, a decrease in the marker from the initial time to the second time may be indicative of a particular diagnosis, or a particular prognosis.

The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples. Preferably, the sample is a blood sample, most preferably a serum sample or a plasma sample.

The term “patient” as used herein refers to a living human or non-human organism that is receiving medical care or that should receive medical care due to a disease. This includes persons with no defined illness who are being investigated for signs of pathology. Thus, the methods and assays described herein are applicable to both human and veterinary disease.

The term “correlating,” as used herein in reference to the use of diagnostic and prognostic markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a panel of marker levels is correlated to a global probability or a particular outcome.

Dyspnea or dyspnoea or shortness of breath (SOB) was defined by the American Thoracic Society as the “subjective experience of breathing discomfort that consists of qualitatively distinct sensations that vary in intensity” (American Heart Society 1999. Am J Respir Crit Care Med 159:321-40). It is a common symptom of numerous medical disorders, particularly those involving the cardiovascular and respiratory systems. This includes obstructive lung diseases (e.g. bronchitis, COPD), pneumonia, sepsis, cardiomyopathy, heart failure and ischemic heart disease. Patients with SOB resulting from trauma, or obvious myocardial infarction (MI) are not subject of the present invention.

Acute heart failure (AHF) is defined as the rapid onset of symptoms and signs secondary to abnormal cardiac function. It may occur with or without previous cardiac disease. The cardiac dysfunction can be related to systolic or diastolic dysfunction, abnormalities in cardiac rhythm, or to pre-load and after-load mismatch. AHF can present itself as acute de novo (new onset of AHF in a patient without previously known cardiac dysfunction) or acute decompensation of chronic heart failure (Nieminen et al. 2005. Eur Heart J 26: 384-416; Dickstein et al. 2008. Eur Hear J 29: 2388-442).

ADM in the context of the present invention relates to adrenomedullin or fragments thereof or precursors or fragments thereof. A preferred fragment of a precursor of ADM is mid-regional proADM (MR-proADM). ProADM₂₄₋₇₁ (or PreproADM₄₅₋₉₂) is a particularly preferred marker peptide in the context of the present invention.

“Fragments” of the marker peptides relate to fragments of at least 12 amino acids in length, preferably at least six amino acid residues in length.

The term “level” in the context of the present invention relates to the concentration (preferably expressed as weight/volume; w/v) of marker peptides in a sample taken from a patient.

The term “outcome” herein relates for instance to the survival of the patient after a defined time, e.g. after 3 days, 5 days, 10 days, 14 days, 20 days, 3 weeks, 4 weeks, 30 days, 45 days, 60 days, 90 days, 3 months, 6 months, 1 year, preferably 30 days.

The term “functional outcome” in the context of the present invention relates to the degree of severity of the disease, i.e. the state of health the patient after a defined time, e.g. 3 days, 5 days, 10 days, 14 days, 20 days, 3 weeks, 4 weeks, 30 days, 45 days, 60 days, 90 days, 3 months, 6 months, 1 year, preferably 30 days.

Determining (or measuring or detecting) the level of a marker peptide herein is performed using a detection method and/or a diagnostic assay as explained below.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “capture molecules” are molecules which may be used to bind target molecules or molecules of interest, i.e. analytes (i.e. in the context of the present invention the cardiovascular peptide(s)), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, capture molecules may for instance be selected from the group comprising a nucleic acid molecule, a carbohydrate molecule, a RNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

The assays can be homogenous or heterogeneous assays, competitive and non-competitive sandwich assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person. (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134), incorporated herein by reference).

In a particularly preferred embodiment the assay comprises two capture molecules, preferably antibodies which are both present as dispersions in a liquid reaction mixture, wherein a first labeling component is attached to the first capture molecule, wherein said first labeling component is part of a labeling system based on fluorescence- or chemiluminescence-quenching or amplification, and a second labeling component of said marking system is attached to the second capture molecule, so that upon binding of both capture molecules to the analyte a measurable signal is generated that allows for the detection of the formed sandwich complexes in the solution comprising the sample.

Even more preferred, said labeling system comprises rare earth cryptates or rare earth chelates in combination with a fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in Kirk-Othmer, Encyclopedia of chemical technology, 4^{th} ed., executive editor, J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters.

The levels, i.e. the concentrations, of the one or more marker peptides (or fragments thereof or precursors or fragments thereof) in the sample of the patient are attributed to the prognosis of an outcome or assessing the risk for the patient. For instance, concentrations of the marker peptide above a certain threshold value are indicative for an adverse outcome or an elevated risk for the patient. Such threshold values are preferably in the range of from about 0.5 to 5.0 pmol/l, more preferably 1.0 to 2.5 pmol/l, most preferably 1.985 nmol/l for MR-proADM and in the range of from about 250 to 15000 pg/ml, more preferably 5000 to 15000 pg/ml, most preferably 10472 pg/ml for NT-proBNP.

In another embodiment of the invention, the risk and/or outcome for a patient is determined by relating the patient's individual level of marker peptide to certain percentiles (e.g. 97.5^{th} percentile) of a healthy population.

Kaplan-Meier estimators may be used for the assessment or prediction of the outcome or risk (e.g. morbidity) of a patient.

### Sequences

The amino acid sequence of the precursor peptide of Adrenomedulin (pre-pro-Adrenomedullin) is given in SEQ ID NO:1. Pro-Adrenomedullin relates to amino acid residues 22 to 185 of the sequence of pre-pro-Adrenomedullin. The amino acid sequence of pro-Adrenomedullin (pro-ADM) is given in SEQ ID NO:2. The pro-ADM N-terminal 20 peptide (PAMP) relates to amino acid residues 22-41 of pre-proADM. The amino acid sequence of PAMP is given in SEQ ID NO:3. MR-pro-Adrenomedullin (MR-pro-ADM) relates to amino acid residues 45-92 of pre-pro-ADM. The amino acid sequence of MR-pro-ADM is provided in SEQ ID NO:4. The amino acid sequence of mature Adrenomedullin (ADM) is given in SEQ ID NO:5.

The sequence of the 134 amino acid precursor peptide of brain natriuretic peptide (pre-pro-BNP) is given in SEQ ID NO:6. Pro-BNP relates to amino acid residues 27 to 134 of pro-pro-BNP. The sequence of pro-BNP is shown in SEQ ID NO:7. Pro-BNP is cleaved into N-terminal pro-BNP (NT-pro-BNP) and mature BNP. NT-pro-BNP comprises the amino acid residues 27 to 102 and its sequence is shown in SEQ ID NO:8. The SEQ ID NO:9 shows the sequence of BNP comprising the amino acid residues 103 to 134 of the pre-pro-BNP peptide.

SEQ ID NO: 1 (amino acid sequence of pre-pro-ADM):

| | |
|---|---|
| 1 | MKLVSVALMY LGSLAFLGAD TARLDVASEF RKKWNKWALS |
| | RGKRELRMSS |
| 51 | SYPTGLADVK AGPAQTLIRP QDMKGASRSP EDSSPDAARI |
| | RVKRYRQSMN |
| 101 | NFQGLRSFGC RFGTCTVQKL AHQIYQFTDK DKDNVAPRSK |
| | ISPQGYGRRR |
| 151 | RRSLPEAGPG RTLVSSKPQA HGAPAPPSGS APHFL |

SEQ ID NO: 2 (amino acid sequence of pro-ADM):

| | |
|---|---|
| 1 | ARLDVASEFR KKWNKWALSR GKRELRMSSS YPTGLADVKA |
| | GPAQTLIRPQ |
| 51 | DMKGASRSPE DSSPDAARIR VKRYRQSMNN FQGLRSFGCR |
| | FGTCTVQKLA |
| 101 | HQIYQFTDKD KDNVAPRSKI SPQGYGRRRR RSLPEAGPGR |
| | TLVSSKPQAH |
| 151 | GAPAPPSGSA PHFL |

SEQ ID NO: 3 (amino acid sequence of pro-ADM N20):

| | |
|---|---|
| 1 | ARLDVASEFR KKWNKWALSR |

SEQ ID NO: 4 (amino acid sequence of MR-pro-ADM):

| | |
|---|---|
| 1 | ELRMSSSYPT GLADVKAGPA QTLIRBQDMK GASRSPEDSS |

SEQ ID NO: 5 (amino acid sequence of ADM):

| | |
|---|---|
| 1 | YRQSMNNFQG LRSFGCRFGT CTVQKLAHQI YQFTDKDKDN |
| | VAPRSKISPQ |
| 51 | GY |

SEQ ID NO: 6 (amino acid sequence of pre-pro-BNP):

| | |
|---|---|
| 1 | MDPQTAPSRA LLLLLFLHLA FLGGRSHPLG SPGSASDLET |
| | SGLQEQRNHL |
| 51 | QGKLSELQVE QTSLEPLQES PRPTGVWKSR EVATEGIRGH |
| | RKMVLYTLRA |
| 101 | PSPKMVQGS GCFGRKMDRI SSSSGLGCKV LRRH |

SEQ ID NO: 7 (amino acid sequence of pro-BNP):

| | |
|---|---|
| 1 | HPLGSPGSAS DLETSGLQEQ RNHLQGKLSE LQVEQTSLEP |
| | LQESPRPTGV |
| 51 | WKSREVATEG IRGHRKMVLY TLRAPRSPKM VQGSGCFGRK |
| | MDRISSSSGL |
| 101 | GCKVLRRH |

SEQ ID NO: 8 (amino acid sequence of NT-pro-BNP)

| | |
|---|---|
| 1 | HPLGSPGSAS DLETSGLQEQ RNHLQGKLSE LQVEQTSLEP |
| | LQESPRPTGV |
| 51 | WKSREVATEG IRGHRKMVLY TLRAPR |

SEQ ID NO: 9 (amino acid sequence of BNP):

| | |
|---|---|
| 1 | SPKMVQGSGC FGRKMDRISS SSGLGCKVLR RH |

### DESCRIPTION OF DRAWINGS

FIG. 1: Survival rates plotted for the four quartiles of MR-proADM levels in AHF patients.

FIG. 2: Survival rates plotted for the four quartiles of MR-proADM levels in AHF patients, quartiles 1 to 3 have been combined.

FIG. 3: Survival rates plotted for the four quartiles of BNP levels in AHF patients.

FIG. 4: Survival rates plotted for the four quartiles of BNP levels in AHF patients, quartiles 1 to 3 have been combined.

FIG. 5: Survival rates plotted for the four quartiles of NT-proBNP levels in AHF patients.

FIG. 6: Survival rates plotted for the four quartiles of NT-proBNP levels in AHF patients, quartiles 1 to 3 have been combined.

FIG. 7: Area under the curve (AUC) from ROC plots for the markers BNP, NT-proBNP and MR-proADM on different days.

FIG. 8: ROC plot for the markers BNP, NT-proBNP and MR-proADM.

FIG. 9: Survival (cumulative survival depending on the day) in patients with and without AHF plotted for MR-proADM above and below the threshold of 1.985 pmol/L.

FIG. 10: Summary of patients.

FIG. 11: SEQ ID NO:1 (amino acid sequence of pre-pro-ADM)

FIG. 12: SEQ ID NO:2 (amino acid sequence of pro-ADM)

FIG. 13: SEQ ID NO:3 (amino acid sequence of pro-ADM N20)

FIG. 14: SEQ ID NO:4 (amino acid sequence of MR-pro-ADM)

FIG. 15: SEQ ID NO:5 (amino acid sequence of ADM)

FIG. 16: SEQ ID NO:6 (amino acid sequence of pre-pro-BNP)

FIG. 17: SEQ ID NO:7 (amino acid sequence of pro-BNP)

FIG. 18: SEQ ID NO:8 (amino acid sequence of NT-pro-BNP)

FIG. 19: SEQ ID NO:9 (amino acid sequence of BNP)

### EXAMPLES

### Example 1

### Clinical Study: 15 Enrolling Centers—Recruiting 1641 Patients with Shortness of Breath (SoB) and Acute Heart Failure (AHF)

TABLE 1
Summary of patients

| | Breathing Not | |
|---|---|---|
| | Properly | BACH |
| Characteristics | | |
| TOTAL ENROLLMENT | 1,586 | 1,641 |
| Age | 64 +/− 17 | 64 +/− 17 |
| Sex Male (%) | 56 | 52 |
| Sex Female (%) | 44 | 48 |
| History (%) | | |
| CHF | 33 | 36 |
| AMI | 27 | 19 |
| COPD | 41 | 30 |
| Diabetes | 25 | 29 |

TABLE 2
Additional baseline data of patients

| | Non-AHF | | AHF | | |
|---|---|---|---|---|---|
| Variable | Mean | SD | Mean | SD | p |
| Heart Rate (bpm) | 93 | 22 | 89 | 25 | 0.005 |
| Temperature (C.) | 36.8 | 0.7 | 36.7 | 0.7 | 0.039 |
| Systolic BP (mmHg) | 140 | 27 | 143 | 32 | 0.027 |
| Diastolic BP (mmHg) | 80 | 16 | 83 | 19 | <0.001 |
| BMI (kg/m2) | 30 | 9 | 29 | 8 | 0.035 |

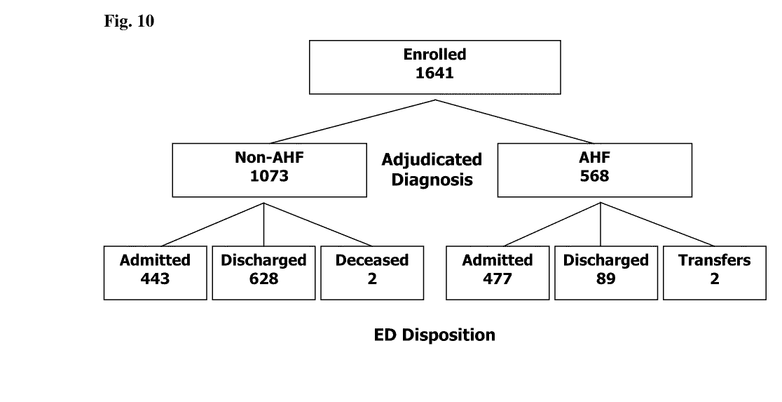

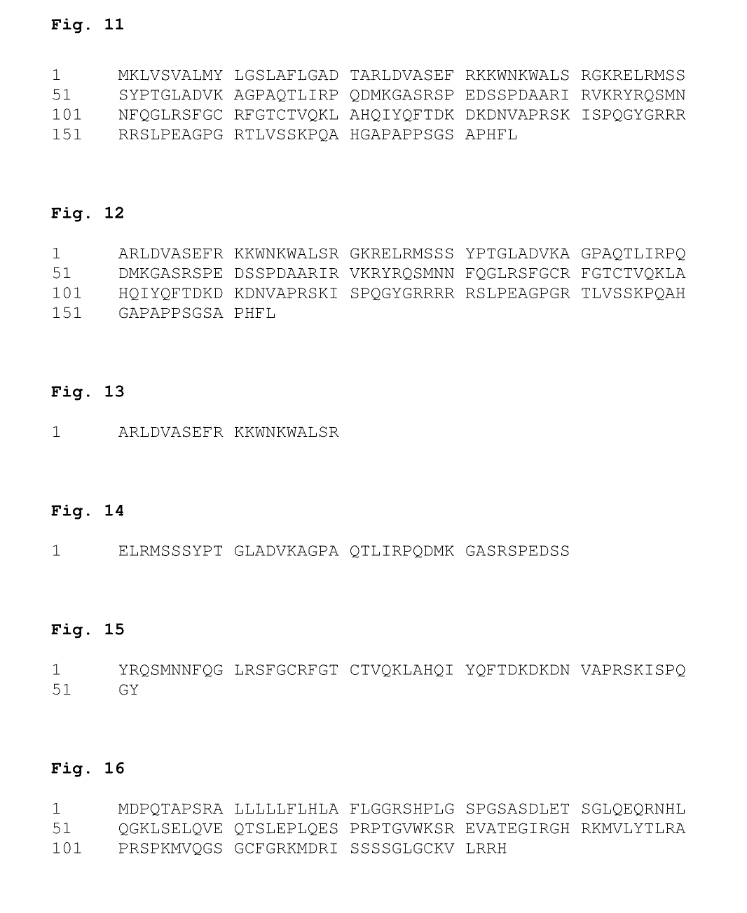

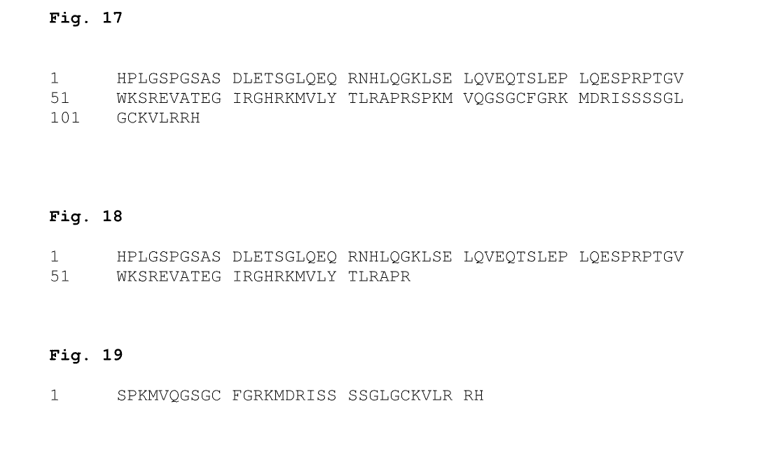

Also see FIG. 10 for summary of patients.

### Study Particulars:

Patients included who presented to emergency department (ED) with SoB not from trauma, or obvious myocardial infarction (MI), and not on dialysis.

After consenting, MD assessment of probability of heart failure and/or pneumonia.

Two independent cardiologists agreed on final diagnosis following discharge.

Follow-up for 90 days for survival; Outcome “All cause mortality within 90 days”.

Survival in AHF—Results of Cox Regression with Continuous Predictors:

| | | | |
|---|---|---|---|
| Diagnostic | MR-proADM 73.5% | vs BNP 60.8% | (p < 0.001) |
| Accuracy: | | vs NT-proBNP 63.6% | (p < 0.001) |

TABLE 3
MR-proADM is superior to BNP and NT-proBNP for
predicting 90-day mortality (Cox regression).

| Predictor (univariate) | Chi² Statistic | p | c index |
|---|---|---|---|
| log MR-proADM | 31.0 | <0.001 | 0.669 |
| log BNP | 7.1 | 0.008 | 0.596 |
| log NT-proBNP | 17.1 | <0.001 | 0.654 |

TABLE 4
MR-proADM adds significantly to BNP or NT-proBNP,
however neither BNP nor NT-proBNP add to MR-proADM

| | Chi² Statistic | p |
|---|---|---|
| adding MR-proADM to BNP | 23.9 | <0.0001 |
| adding MR-proADM to NT-proBNP | 15.3 | <0.0001 |
| adding BNP to MR-proADM | 0.0 | 0.906 |
| adding NT-proBNP to MR-proADM | 1.1 | 0.291 |

### Survival in AHF—Results of Multivariable Cox Regression:

TABLE 5
MR-proADM is more important than BNP in
survival models using Cox Regression.

| Predictor (multivariable) | HR | 95% CI | p |
|---|---|---|---|
| log MR-proADM | 14.0 | 5.0-39.4 | <0.001 |
| log BNP | 1.0 | 0.5-2.0 | 0.906 |

TABLE 6
MR-proADM is more important than NT-proBNP
in survival models using Cox Regression.

| Predictor (multivariable) | HR | 95% CI | p |
|---|---|---|---|
| log MR-proADM | 10.4 | 3.3-32.7 | <0.001 |
| log NT-proBNP | 1.4 | 0.7-2.6 | 0.295 |

Survival in AHF—Cox Models with Troponin Elevation

Troponin values were available in 511 of 568 HF patients in 107 (20.9%) patients they were elevated.

TABLE 7
In models with 3 markers, Troponin & MR-proADM
provide prognostic utility, but BNP does not.

| Predictor (multivariable) | HR | 95% CI | p |
|---|---|---|---|
| log MR-proADM | 8.5 | 2.7-26.5 | <0.001 |
| log BNP | 0.9 | 0.5-1.9 | 0.812 |
| Elevated Tn | 2.6 | 1.5-4.5 | <0.001 |

TABLE 8
In models with 3 markers, Troponin & MR-proADM provide
prognostic utility, but NT-proBNP does not.

| Predictor (multivariable) | HR | 95% CI | p |
|---|---|---|---|
| log MR-proADM | 7.5 | 2.1-26.4 | <0.001 |
| log NT-proBNP | 1.1 | 0.6-2.2 | 0.295 |
| Elevated Tn | 2.6 | 1.5-4.4 | <0.001 |

Clinical lab Troponin values (TnI or TnT) were judged as elevated if above the local normal range.

TABLE 9
MR-proADM adds significantly to Troponin and
Tropoinin adds significantly to MR-proADM

| | Chi² Statistic | p |
|---|---|---|
| adding Troponin to MR-proADM | 10.33 | 0.0013 |
| adding MR-proADM to Troponin | 16.41 | <0.0001 |

### Survival in AHF—MR-proADM Quartiles

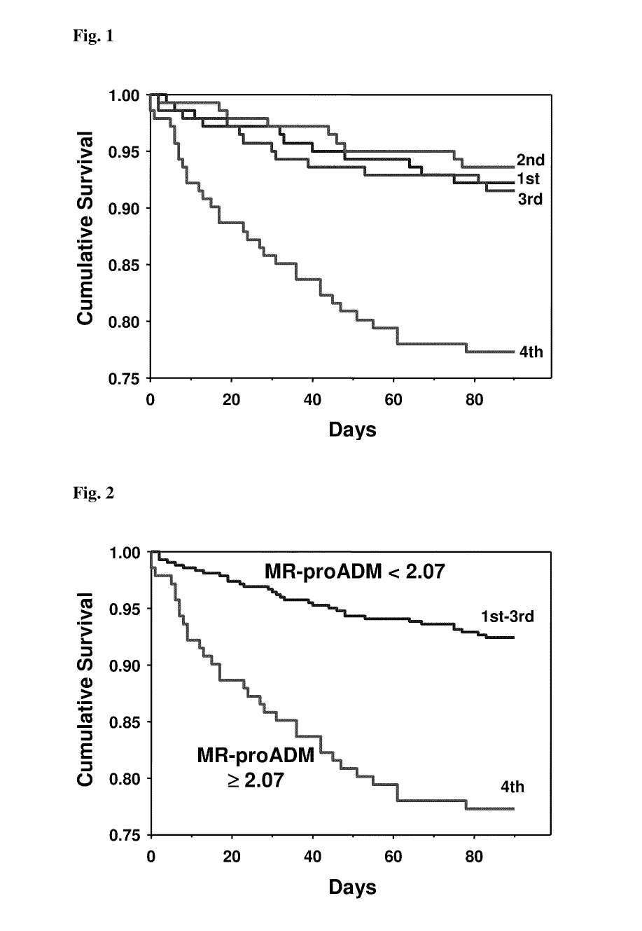

Risk is great in the highest quartile of MR-proADM, see FIGS. 1 and 2 and tables 10 and 11.

TABLE 10

| | | | |
|---|---|---|---|
| Quartile | HR | 95% CI | p |

| | | |
|---|---|---|
| 1st | 1 | reference |

| | | | |
|---|---|---|---|
| 2nd | 0.8 | 0.3-2.0 | 0.640 |
| 3rd | 1.1 | 0.5-2.5 | 0.822 |
| 4th | 3.2 | 1.6-6.4 | 0.001 |

TABLE 11

| | | | |
|---|---|---|---|
| Quartile | HR | 95% CI | p |

| | | |
|---|---|---|
| 1st-3rd | 1 | reference |

| | | | |
|---|---|---|---|
| 4th | 3.3 | 2.0-5.4 | <0.001 |

### Survival in AHF—BNP Quartiles

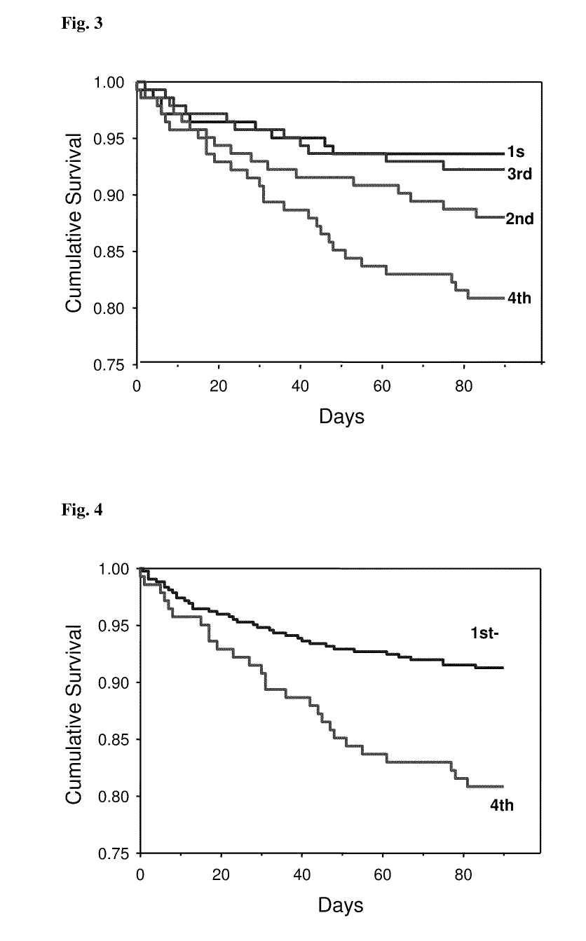

Risk is great in the highest quartile of BNP, see FIGS. 3 and 4 and tables 12 and 13.

TABLE 12

| | | | |
|---|---|---|---|
| Quartile | HR | 95% CI | p |

| | | |
|---|---|---|
| 1st | 1 | reference |

| | | | |
|---|---|---|---|
| 2nd | 1.9 | 0.9-4.3 | 0.116 |
| 3rd | 1.2 | 0.5-2.9 | 0.668 |
| 4th | 3.2 | 1.5-6.7 | 0.003 |

TABLE 13

| | | | |
|---|---|---|---|
| Quartile | HR | 95% CI | p |

| | | |
|---|---|---|
| 1st-3rd | 1 | reference |

| | | | |
|---|---|---|---|
| 4th | 2.3 | 1.4-3.8 | <0.001 |

### Survival in AHF—NT-proBNP Quartiles

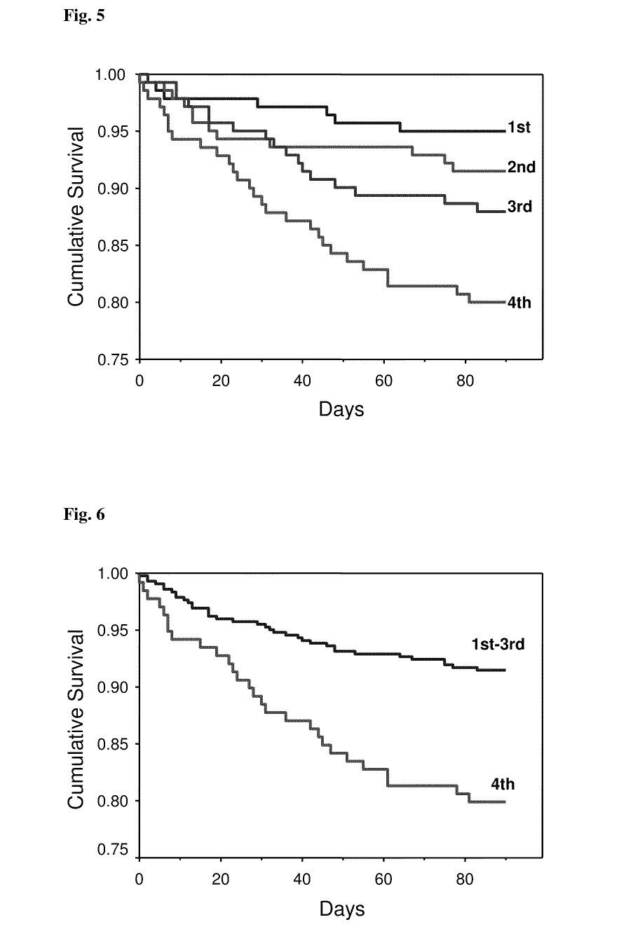

Risk is great in the highest quartile of NT-proBNP, see FIGS. 5 and 6 and tables 14 and 15.

TABLE 14

| | | | |
|---|---|---|---|
| Quartile | HR | 95% CI | p |

| | | |
|---|---|---|
| 1st | 1 | reference |

| | | | |
|---|---|---|---|
| 2nd | 1.7 | 0.7-4.4 | 0.247 |
| 3rd | 2.5 | 1.0-6.0 | 0.043 |
| 4th | 4.3 | 1.9-9.9 | <0.001 |

TABLE 15

| | | | |
|---|---|---|---|
| Quartile | HR | 95% CI | p |

| | | |
|---|---|---|
| 1st-3rd | 1 | reference |

| | | | |
|---|---|---|---|
| 4th | 2.5 | 1.5-4.1 | <0.001 |

### Survival in AHF—Area Under the Roc Curve Comparison

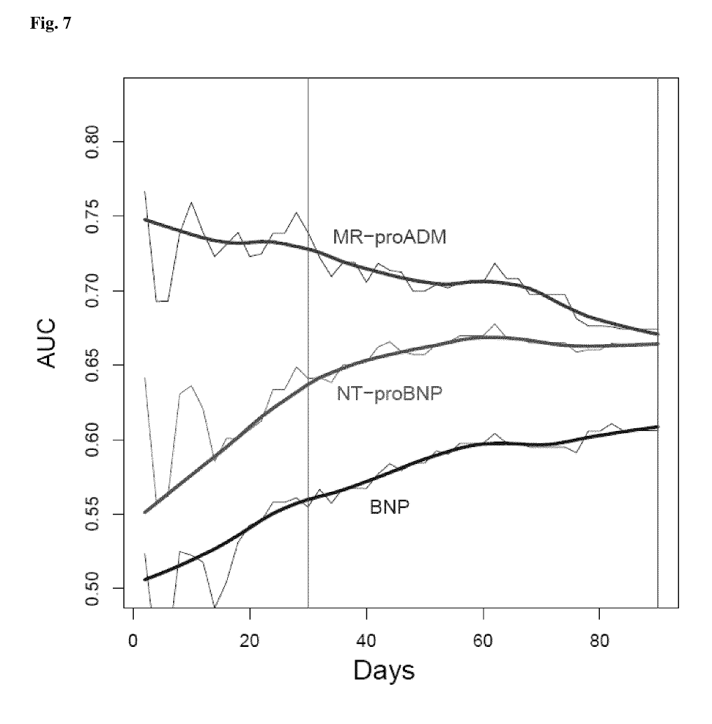

MR-proADM predicts short term (30 day) survival exceptionally well, see FIG. 7 and table 16.

TABLE 16

| AUC | 30 days | 90 days |
|---|---|---|
| MR-proADM | 0.739 | 0.674 |
| NT-proBNP | 0.641 | 0.664 |
| BNP | 0.555 | 0.606 |

Survival in all Patients with SoB—Utility of MR-proADM

TABLE 17
Cox Regression Analysis, MR-proADM
performs well in all SoB. patients.

| Predictor (univariate) | Chi² Statistic | p | c index |
|---|---|---|---|
| log MR-proADM | 129.5 | <0.0001 | 0.755 |
| log BNP | 60.1 | <0.0001 | 0.691 |
| log NT-proBNP | 83.7 | <0.0001 | 0.721 |

TABLE 18
Cox Regression Analysis, MR-proADM
is superior to BNP and NT-proBNP.

| | Chi² Statistic | p |
|---|---|---|
| adding MR-proADM to BNP | 69.4 | <0.0001 |
| adding MR-proADM to NT-proBNP | 46.6 | <0.0001 |
| adding BNP to MR-proADM | 0.1 | 0.731 |
| adding NT-proBNP to MR-proADM | 1.5 | 0.229 |

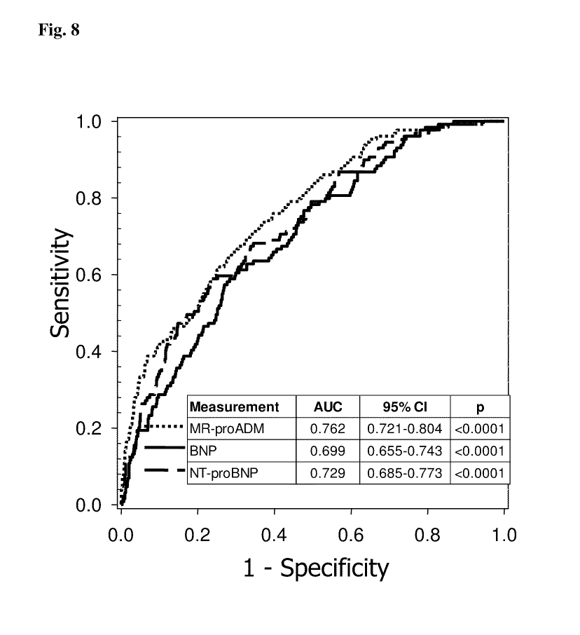

A corresponding ROC plot is shown in FIG. 8.

Survival in Patients without AHF—Utility of MR-proADM

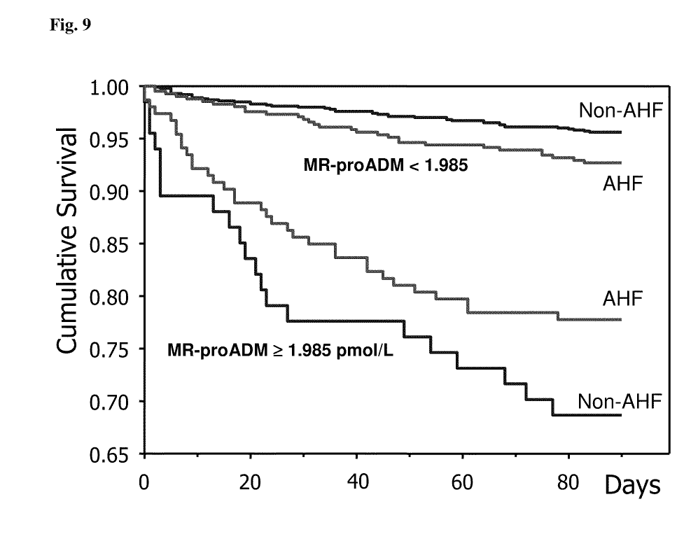

Elevated MR-proADM is strongly prognostic in patients with and without AHF—even more so in non-AHF than in AHF (interaction p=0.005). See appended FIG. 9 and tables 19 and 20.

TABLE 19
AHF patients

| | AUC | optimal cut point |
|---|---|---|
| | (90 days) | from ROC |
| MR-proADM | 0.674 | 1.985 pmol/l |

TABLE 20
Error! Bookmark not defined.

| | | | | |
|---|---|---|---|---|
| Diagnosis | MR-proADM | HR | 95% CI | P |

| | | | |
|---|---|---|---|
| Non-AHF | low < 1.985 | 1 | reference |

| | | | | |
|---|---|---|---|---|
| | high ≧ 1.985 | 8.6 | 5.1-14.4 | <0.001 |
| AHF | low < 1.985 | 1.7 | 1.1-2.7 | 0.027 |
| | high ≧ 1.985 | 5.7 | 3.6-8.9 | <0.001 |

### Summary of Study:

MR-proADM is a strong prognosticator in patients with AHF and in patients presenting with SoB.
MR-proADM is superior to BNP or NT-proBNP for predicting 90-day mortality, both in AHF as well as in all ED patients with SoB.
MR-proADM is particularly strong in predicting short-term prognosis within 4 weeks after assessment.
All these results are unaffected by adjustment for Troponin.
MR-proADM can significantly improve risk stratification over BNP or NT-proBNP.

Assessment of MR-proADM can help to identify patients who should “move to the front of the line” of medical care.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding European application No. 08 16 8816.0, filed Nov. 11, 2008, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for the prognosis of an outcome or assessing the risk of a patient suffering from heart failure and/or shortness of breath, comprising the following steps:
a. providing a sample from said patient,
b. determining the level of ADM or fragments thereof or its precursor or fragments thereof in said sample,
c. correlating the level of said ADM or fragments thereof or its precursor or fragments thereof with the prognosis of an outcome or assessing the risk for said patient.

2. A method for the prognosis of an outcome or assessing the risk of a patient suffering from heart failure and/or shortness of breath, comprising the following steps:
a. providing a sample from said patient,
b. determining the level of ADM or fragments thereof or its precursor or fragments thereof in said sample,
c. determining the level of BNP or fragments thereof or its precursor or fragments thereof, in said sample,
d. correlating the level of said ADM or fragments thereof or its precursor or fragments thereof and BNP or fragments thereof or its precursor or fragments thereof with the prognosis of an outcome or assessing the risk for said patient.

3. A method according to claim 1, wherein said outcome or said risk regards survival and/or a functional outcome.

4. A method according to claim 1, wherein the outcome after 3 days, 5 days, 10 days, 14 days, 20 days, 3 weeks, 4 weeks, 30 days, 90 days, 3 months, 6 months, 1 year, preferably 30 days is predicted.

5. A method for the stratification of a patient into risk groups wherein said patient is suffering from heart failure and/or shortness of breath and said method comprising the steps according to claim 1.

6. A method according to claim 1, wherein ADM or fragments thereof or its precursor or fragments thereof is MR-proADM.

7. A method according to claim 1, wherein BNP or fragments thereof or its precursor or fragments thereof is NT-proBNP.

8. A method according to claim 1, wherein the level of ADM or fragments thereof or its precursor or fragments thereof and BNP or fragments thereof or its precursor or fragments thereof are differently weighted.

9. A method according to claim 1, wherein the functional outcome is determined as ranking or the degree of severity of the outcome.

10. A method for the prognosis of an outcome or assessing the risk of a patient suffering from heart failure and/or shortness of breath according to claim 1, wherein the levels of ADM or fragments thereof or its precursor or fragments thereof and BNP or fragments thereof or its precursor or fragments thereof either alone or in conjunction with other prognostically useful laboratory or clinical parameters are used for the prediction of an outcome or assessing the risk of a patient suffering from heart failure and/or shortness of breath by a method which may be selected from the following alternatives:
Comparison with the medians of the levels of ADM or fragments thereof or its precursor or fragments thereof and BNP or fragments thereof or its precursor or fragments thereof in an ensemble of pre-determined samples in a population of patients suffering from heart failure and/or shortness of breath,
Comparison with a quantile of the levels of ADM or fragments thereof or its precursor or fragments thereof and BNP or fragments thereof or its precursor or fragments thereof in an ensemble of pre-determined samples in a population of patients suffering from heart failure and/or shortness of breath,
Calculation based on Cox Proportional Hazards analysis or by using Risk index calculations such as the NRI (Net Reclassification Index) or the IDI (Integrated Discrimination Index).

11. A method according to claim 1, wherein additionally at least one clinical parameter is determined selected from the group comprising: age, gender, systolic blood pressure, diastolic blood pressure, antihypertensive treatment, body mass index, heart rate, temperature, presence of diabetes mellitus, current smoking habits.

12. A method according to claim 1, wherein additionally at least one other laboratory parameter is determined selected from the group comprising troponin, myeloperoxidase, CRP, neopterin, GDF-15, ST2, cystatin-C, as well as the following peptides in form of their mature peptides, precursors, pro-hormones and associated prohormone fragments: atrial natriuretic peptide, endothelins, vasopressin.

13. Kit for the prognosis of an outcome or assessing the risk of a patient suffering from heart failure and/or shortness of breath comprising at least one capture probe directed against ADM or fragments thereof or precursors or fragments thereof and at least one capture probe directed against BNP or fragments thereof or precursors or fragments thereof.

14. Kit according to claim 13, wherein the capture probe directed against ADM or fragments thereof or precursors or fragments thereof is directed against MR-proADM.

15. Kit according to claim 13, wherein the capture probe directed against BNP or fragments thereof or precursors or fragments thereof is directed against NT-proBNP.

16. Method according to claim 1 for the prognosis of an outcome or assessing the risk of a patient suffering from heart failure and/or shortness of breath.

17. Method according to claim 1 for monitoring of the therapy in a patient suffering from heart failure and/or shortness of breath.

18. Method for the prognosis of an outcome or assessing the risk of a patient suffering from heart failure and/or shortness of breath using a kit of claim 13.

19. Method for monitoring of the therapy in a patient suffering from heart failure and/or shortness of breath using a kit of claim 13.


SEQUENCE LISTING (9 sequences)

SEQ ID NO: 1 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 20 aa, Homo sapiens
ARLDVASEFRKKWNKWALSR

SEQ ID NO: 4 - protein, 40 aa, Homo sapiens
ELRMSSSYPTGLADVKAGPAQTLIRPQDMKGASRSPEDSS

SEQ ID NO: 5 - protein, 52 aa, Homo sapiens
YRQSMNNFQGLRSFGCRFGTCTVQKLAHQIYQFTDKDKDNVAPRSKISPQGY

SEQ ID NO: 6 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 7 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 8 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 9 - protein, 32 aa, Homo sapiens
SPKMVQGSGCFGRKMDRISSSSGLGCKVLRRH